(19) United States
(12) Patent Application Publication
(10) Pub. No.: US 2025/0050343 A1
(43) Pub. Date: Feb. 13, 2025

(54) BIOLOGICAL TESTING KITS AND METHODS FOR USING AND MAKING THE SAME

(71) Applicant: SILGAN DISPENSING SYSTEMS CORPORATION, Richmond, VA (US)

(72) Inventors: BRANDON RAMSUER, HENRICO, VA (US); CONNIE TRAN, RICHMOND, VA (US)

(21) Appl. No.: 18/230,856

(22) Filed: Aug. 7, 2023

(51) Int. Cl. B01L 3/00; A61B 10/02

(52) U.S. Cl. CPC B01L 3/505; A61B 10/02; B01L 2200/0689; B01L 2200/16; B01L 2300/042; B01L 2300/0832

(57) ABSTRACT

A blow molded vial and a testing kit including a blow molded vial in which the vial includes one or two potential openings, capable of being opened and rescaled during the collection of a sample and further opened to dispense a sample and reagent or carrier fluid from within an inner space of the blow molded vial.

### BACKGROUND OF THE INVENTION

### Field of the Invention

Embodiments of the invention relate to testing kits, methods of using testing kits, and methods of making testing kits; more particularly, embodiments of the invention relate to testing vials used with testing kits and methods for making, filling, and using such vials.

### Description of Related Art

Biological testing kits are well known and there are many different types and examples of such kits. Many biological testing kits utilize glass vials to contain a reagent or carrier fluid into which a biological sample may be transferred or dissolved. For example, in certain COVID-19 tests, a reagent is contained within a sealed vial and a nasal swab from a user is inserted into the vial and allowed to sit so that the biological sample from the swab will transfer to the reagent in the vial. The vial and swab may need to be set aside, allowing the swab to soak in the reagent. Once the biological sample has been sufficiently transferred to the reagent—for example, by immersing a swab with biological sample in the vial and reagent for a calculated amount of time—the swab may be removed and discarded and the sample-containing reagent further tested.

In some instances, a sample-containing reagent contained within the vial may be poured out of the vial for testing. In other instances, a delivery tool—such as a pipette—may be used to remove a portion of a reagent containing sample from a vial for distribution onto a test strip or other carrier from which a sample will be tested.

While these methods provide good results and are adequate for performing tests, it would be beneficial if the amount of material used, and waste generated, could be reduced. Further, it is desirable to have more compact systems and kits that can be easily used in the field or in locations lacking more sophisticated testing facilities, clean rooms, or resources.

### BRIEF SUMMARY OF THE INVENTION

According to certain embodiments of the invention, a biological test kit may include a blow-molded plug top vial containing one or more reagents or fluid samples. The plug top vial may include a blow-molded container having a first end and a second end with a hollow space defined therebetween. The first end may be sealed with a plug. The second end may be sealed with a twist-off or break-away tip. A reagent capable of being used for a biological test or as a carrier fluid for a biological sample may be stored in the plug top vial. The biological test kit may also include a swab.

According to other embodiments of the invention, a biological test kit may include a blow-molded threaded top vial containing one or more reagents or fluid samples. The threaded top vial may include a blow-molded container having a first end and a second end with a hollow space defined therebetween. The first end may include a hollow top portion that may be torn away, twisted, snapped, or otherwise separated from the container. The hollow top portion may include threads within the interior thereof or a snap feature or bayonet feature to allow the top portion to be reattached to the container. A portion of the container adjacent to the hollow top portion may include exterior threads, snap connectors, bayonet connectors, or other connection features to allow reattachment of the hollow top portion to the container after being removed. The second end of the threaded top vial may be sealed with a twist-off or break-away tip that can be removed to expose an interior of the container.

In other embodiments of the invention, a biological test kit may include a blow-molded dropper top vial containing one or more reagents or fluid samples. The dropper top vial may include a blow-molded container having a first end and a second end with a hollow space defined therebetween. The first end may include a blow-molded pipette and closure that may be twisted off, broken off, or otherwise separated from the container to expose the hollow space in the container. The blow-molded pipette may be used to remove liquid—such as the reagent or reagent and mixed sample—from an interior of the container.

According to other embodiments of the invention, a biological test kit may include a blow-molded swab closure having a first end and a second end with a hollow space defined therebetween. The first end may include a removable tab or top that can be twist-off, broken off, or otherwise removed from the container to expose the hollow space therein. The first end may also include threads, snap beads, or other features configured to allow attachment of a closure to the swab closure. The second end of the swab closure may be sealed with a twist-off or break-away tip that can be removed to expose an interior or hollow space within the container. The biological test kit may also include a swab attached to a closure. In some embodiments, the closure may include threads, snap beads, bayonet features, or other closure features that may mate with and seal the closure to the swab closure. For example, a closure with threads may be attached to a swab such that the swab may be inserted into an open first end of the swab closure and the closure threaded onto the swab closure to seal the swab within the hollow space in the swab closure container.

In still other embodiments of the invention, a biological test kit may include a blow-molded vial having a first end with a removable seal fitted thereon such that the removable seal may be removed from the vial, exposing a hollow interior space which may contain a reagent or a fluid. One or more connection features may be present about an end of the vial adjacent to the opening formed by removal of the removable seal. For example, the vial may include threads or snap fitment features adjacent that portion of the vial where the opening is formed after removal of the removable seal. The biological test kit may also include a swab attached to a closure. In some embodiments, the closure may include a fluid flow path through the closure and a flip cap configured to be able to be closed over the fluid flow path, providing a seal therewith. Removal of the flip cap may open the fluid flow path, allowing fluid to flow through the cap.

In use, a vial containing a reagent may be opened by removing the removable seal. A swab attached to a closure may be used to collect a biological sample and then the end of the swab inserted into a hollow space within the vial. The closure may be attached to or sealed with the vial, such as by threaded connections, snap-fit connections, bayonet connections, or other connection features. Once connected, the swab is exposed to a reagent or fluid carrier and sample transferred from the swab to the reagent or fluid carrier. The cap of the closure may then be opened and the combined reagent/sample or carrier fluid/sample mixture dispensed onto a testing strip, carrier plate, or other substrate to be used for testing the biological sample.

According to further embodiments of the invention, any of the plug top vial, threaded top vial, dropper top vial, swab closure and vial, or dispensing closure and vial may be used as part of a biological test process. According to embodiments of the invention, any of the vials or containers may be blow-molded or blow-molded and filled using traditional blow-molding and blow-molding and filling processes. Once filled, the vials may be assembled into or sorted into biological testing kits and utilized. Alternatively, the vials may be distributed for further use alone or along with portions of other items required for sampling, for instance, with a swab or the dispensing swab closure or swab closure of embodiments of the invention.

### BRIEF DESCRIPTION OF THE SEVERAL VIEWS OF THE DRAWINGS

While the specification concludes with claims particularly pointing out and distinctly claiming particular embodiments of the present invention, various embodiments of the invention can be more readily understood and appreciated by one of ordinary skill in the art from the following descriptions of various embodiments of the invention when read in conjunction with the accompanying drawings in which:

FIG. 1 illustrates a vial in use according to various embodiments of the invention;

FIG. 2 illustrates a user swabbing a nose for a biological sample according to various embodiments of the invention;

FIG. 3 illustrates a swab vial combination according to various embodiments of the invention;

FIG. 4 illustrates a swab being discarded according to various embodiments of the invention;

FIG. 5 illustrates a plug top vial in use according to various embodiments of the invention;

FIG. 6 illustrates a tip being removed from a vial according to various embodiments of the invention;

FIG. 7 illustrates a vial dispensing a sample onto a sample slide according to various embodiments of the invention;

FIG. 8 illustrates a vial being discarded according to various embodiments of the invention;

FIG. 9 illustrates a threaded top vial in use according to various embodiments of the invention;

FIG. 10 illustrates a threaded top vial in use according to various embodiments of the invention;

FIG. 11 illustrates threaded top vial in use according to various embodiments of the invention;

FIG. 12 illustrates a tip being removed from a vial according to various embodiments of the invention;

FIG. 13 illustrates a dropper top vial in use according to various embodiments of the invention;

FIG. 14 illustrates a swab in use with a dropper top vial according to various embodiments of the invention;

FIG. 15 illustrates a pipette of a dropper top vial according to various embodiments of the invention being used to collect a sample from the vial;

FIG. 16 illustrates a pipette of a dropper top vial being used to dispense a sample onto a sample slide according to various embodiments of the invention;

FIG. 17 illustrates a vial being opened according to various embodiments of the invention;

FIG. 18 illustrates a swab closure being used with a vial according to various embodiments of the invention;

FIG. 19 illustrates a swab closure being used with a vial according to various embodiments of the invention;

FIG. 20 illustrates a tip being removed from a vial with an attached swab closure according to various embodiments of the invention;

FIG. 21 illustrates the dispensing of a product from a vial with an attached swab closure according to various embodiments of the invention;

FIG. 22 illustrates a vial being opened according to various embodiments of the invention;

FIG. 23 illustrates a dispensing swab closure being used with a vial according to various embodiments of the invention;

FIG. 24 illustrates a dispensing swab closure being attached to a vial according to various embodiments of the invention;

FIG. 25 illustrates a dispensing swab closure being opened according to various embodiments of the invention;

FIG. 26 illustrates a dispensing swab closure being used to dispense a sample from a vial attached to the dispensing swab closure onto a sample slide according to various embodiments of the invention;

FIG. 27 illustrates a plug top vial according to various embodiments of the invention;

FIG. 28 illustrates a threaded top vial according to various embodiments of the invention;

FIG. 29 illustrates a dropper top vial according to various embodiments of the invention;

FIG. 30 illustrates a dual end vial and swab closure according to various embodiments of the invention; and

FIG. 31 illustrates a dropper vial and swab dispenser according to various embodiments of the invention.

### DETAILED DESCRIPTION OF THE INVENTION

According to embodiments of the invention, a testing kit 100 may include a vial and a swab, the vial containing a reagent or fluid carrier used to collect a sample such as a biological sample. Vials, according to various embodiments of the invention, are blow-molded and have different features, providing improved usability for testing kits.

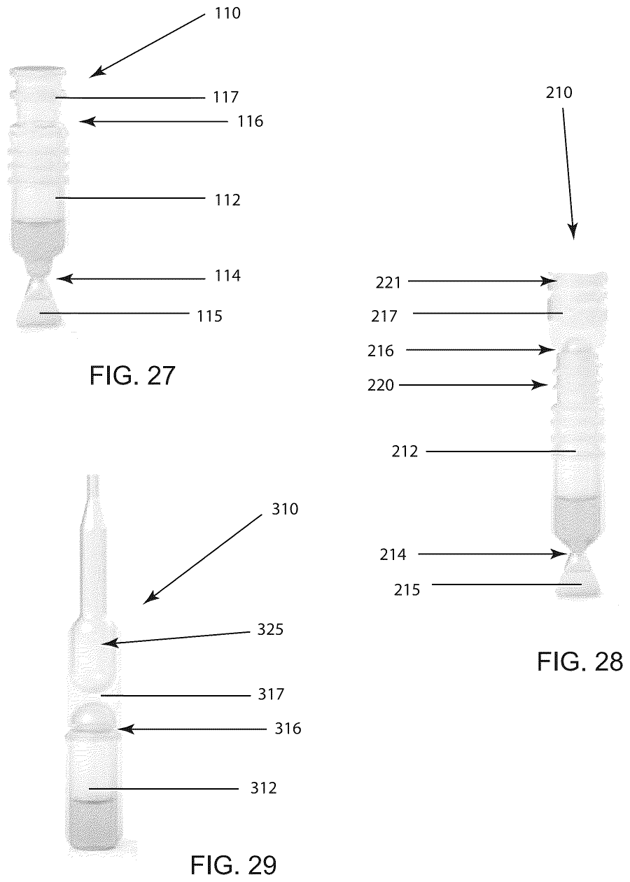

According to a first embodiment of the invention, a testing kit 100 may include a plug top vial 110 and a swab 900. The plug top vial 110 includes a vial body 112 having a second end 114 and a first end 116 as illustrated in FIG. 27. The second end 114 includes a removable or break-away tip 115 and the first end 116 includes a removable plug 117. A hollow space is formed in the plug top vial 110 between the second end 114 and the first end 116. A reagent or fluid carrier may be contained within the plug top vial 110 in the hollow space.

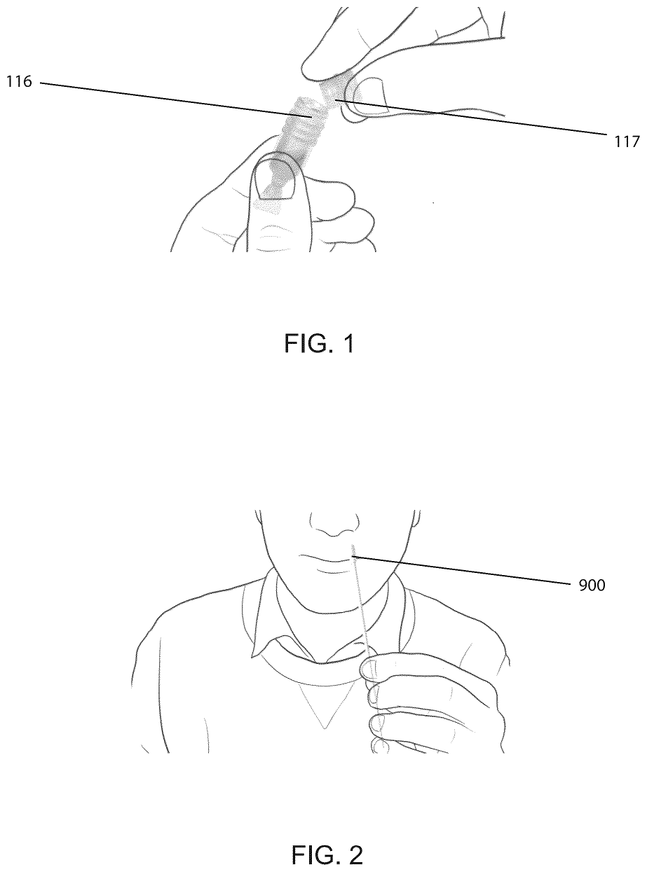

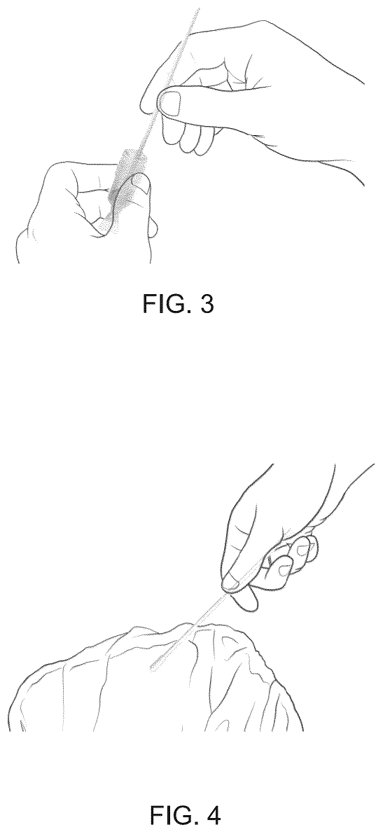

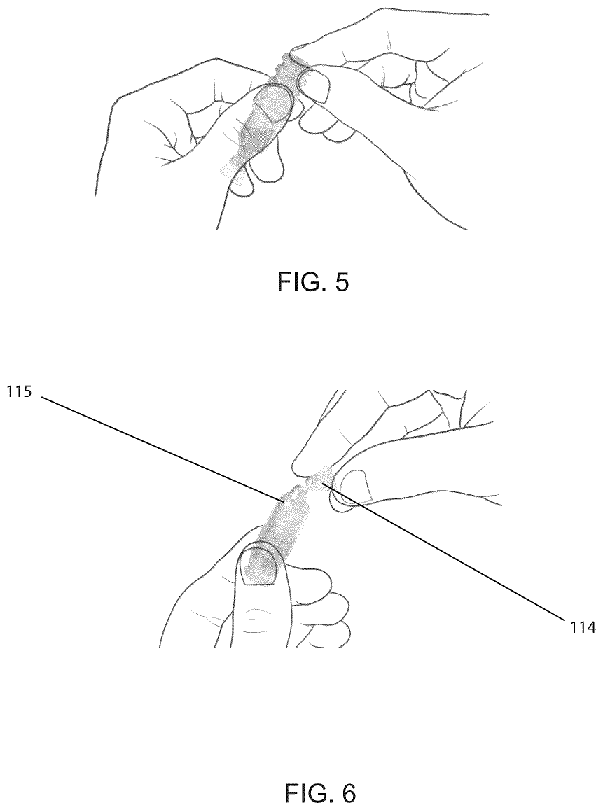

A plug 117 may be snapped off, twisted off, or otherwise removed from the plug top vial 110 as illustrated in FIG. 1. In some embodiments, the joint or material between the vial body 112 and the plug 117 may be scored, thinned, or otherwise shaped to promote the removal of the plug 117 from the vial body 112. Once removed, the first end 116 is open, allowing access to the interior hollow space of the plug top vial 110. A swab 900 may be used to collect a sample from a test subject. For example, a nasal swab 900 may be used to collect a nasal, or nasal mucus sample, from a person as illustrated in FIG. 2. Once a sample is collected, the swab 900 may be inserted into the hollow space of the plug top vial 110 as illustrated in FIG. 3. The swab 900 and sample may contact a reagent or fluid carrier contained in the hollow space of the plug top vial 110, transferring sufficient sample to the reagent or carrier fluid. Once the swab 900 has been exposed to the reagent or carrier fluid for a sufficient amount of time, the swab 900 may be disposed as illustrated in FIG. 4. The plug 117 may then be reinserted into the first end 116 as illustrated in FIG. 5. Insertion of a part of the plug 117 into the first end 116 reseals the vial body 112 such that fluid cannot exit the first end 116 because of the seal between the plug 117 and the first end 116, closing the opening formed by earlier removal of the plug 117. In various embodiments of the invention, the plug 117 end inserted into the open first end 116 is the top of the plug 117 or that portion of the plug 117 that was not originally attached to the plug top vial 110 at the location where it was snapped off, twisted off, or removed from the body of the plug top vial 110.

In some embodiments, the plug 117 end that was originally connected to the vial body 112 may sloped, tapered, or configured such that having been removed from the vial body 112 it can now be reinserted into the first end 116. In other embodiments, the opposite end of the plug 117 may be sloped, tapered, or configured so that it can be inserted into the open first end 116 such that it creates a seal when engaged with the first end 116. In addition, the plug 117 end that is reinserted into the open first end 116 of the plug top vial 110 may include features, such as a snap bead, rim, ridge, or projection, that may interact with an interior portion or feature of the plug top vial 110 adjacent the first end 116 when the plug 117 is inserted into the opening in the first end 116 to assist with scaling the opening former by previous removal of the plug 117.

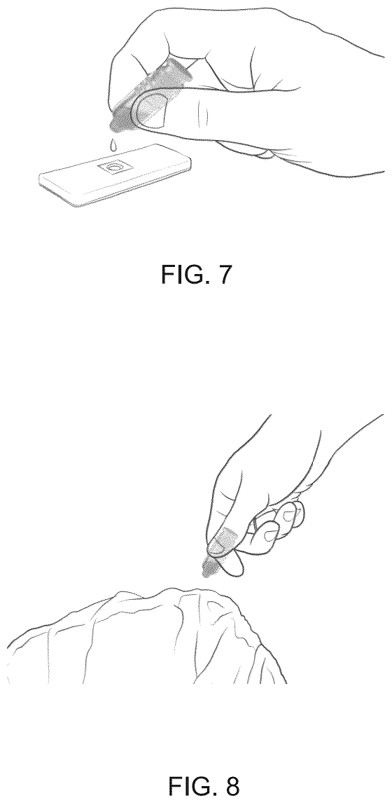

Following sufficient exposure time of the sample to the reagent or carrier fluid in the plug top vial 110, the break-away tip 115 may be removed from the second end 114, creating an opening in the second end 114 as illustrated in FIG. 6. The reagent or carrier fluid carrying the sample may then be dropped or dispensed from the plug top vial 110 through the open second end 114 as illustrated in FIG. 7. In some instances, the sample and reagent or carrier fluid may be dripped onto a sample slide or other substrate for further processing or analysis of the sample dispensed from the plug top vial 110. The plug top vial 110 with any remaining sample may be disposed of as illustrated in FIG. 8.

According to other embodiments of the invention, a threaded top vial 210 as illustrated in FIG. 28 is provided. The threaded top vial 210 includes a vial body 212 having a first end 216 and a second end 214 with the vial body 212 defining a hollow space between the first end 216 and second end 214. A removable top portion 217 is attached at the first end 216 and a break away tip 215 is attached at the second end 214. A reagent or carrier fluid may be contained in the threaded top vial 210 following blow-molding and filling of the threaded top vial 210, for example, through a blow-fill-seal process. Threads 220 or other vial connection features may be formed or present on an exterior surface of the vial body 210 adjacent the first end 216 of the threaded top vial 210. Corresponding threads 221 or other top portion connection features may be formed on an interior portion of the top portion 217 of the threaded top vial 210.

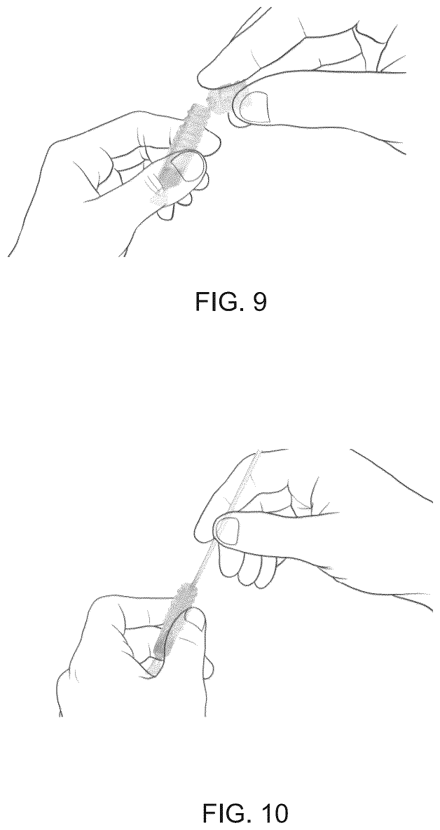

In use, the top portion 217 of the threaded top vial 210 may be twisted off, broken off, or otherwise removed from the vial body 212, opening the first end 216 of the threaded top vial 210 as illustrated in FIG. 9. Once opened, a swab 900 may be used to collect a sample as illustrated in FIG. 2 and the swab 900 may then be inserted in the open first end 216 of the threaded top vial 210 as illustrated in FIG. 10. Once the sample from the swab 900 has been sufficiently exposed to the reagent or carrier fluid in the vial body 212, the swab 900 may be removed and discarded.

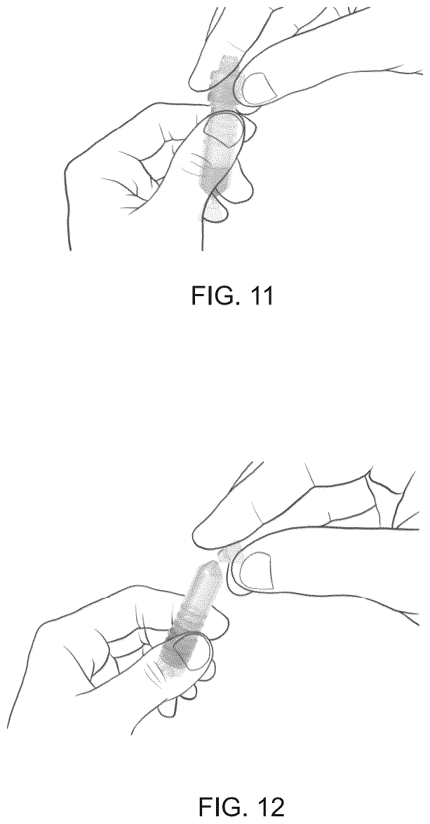

As illustrated in FIG. 11, the open first end 216 of the threaded top vial 210 may be sealed with the top portion 217 of the threaded top vial 210 that was previously removed. According to certain embodiments of the invention, the end of the top portion 217 opposite the original connection to the vial body 212 may be arranged over the first end 216 vial body 212 to engage the threads 220 on the exterior of the vial body 212 and may be screwed or threaded into a sealing position. While the illustrated threaded top vial 210 includes threads to provide a scaling engagement between the top portion 217 and the vial body 212, other attachment features may also be used. For example, the top portion 217 and vial body 212 may include snap fitment features or bayonet features allowing the reconnection and sealing of the top portion 217 with the vial body 212 to close and seal the first end 216 of the threaded top vial 210. Complementary connection features may be provided on the vial body 212 and the top portion 217 to facilitate the closing and sealing of the first end 216 of the threaded top vial 210.

Following sufficient exposure of the sample to the reagent or carrier fluid in the threaded top vial 210, the break-away tip 215 may be twisted off, broken off, or otherwise removed from the threaded top vial 210, opening the second end 214 as illustrated in FIG. 12. The sample mixed with the reagent or carrier fluid may then be dropped or dispensed onto a carrier substrate or slide for further processing or testing procedures.

An alternate embodiment of the invention is illustrated in FIG. 29. A dropper top vial 310 is provided wherein the dropper top vial 310 includes a vial body 312 defining a hollow space or sample container. A first opening 216 in the vial body 312 is sealed with a blow molded dropper 317 or pipette. The blow molded dropper 317 includes an interior space 325 in communication with an opening at an end of the blow molded dropper 317. Squeezing an exterior of the blow molded dropper 317 collapses at least a portion of the interior space 325, eliminating or dispensing product from therein or forcing air out to allow a vacuum force when released such that the dropper can act like a pipette or dropper.

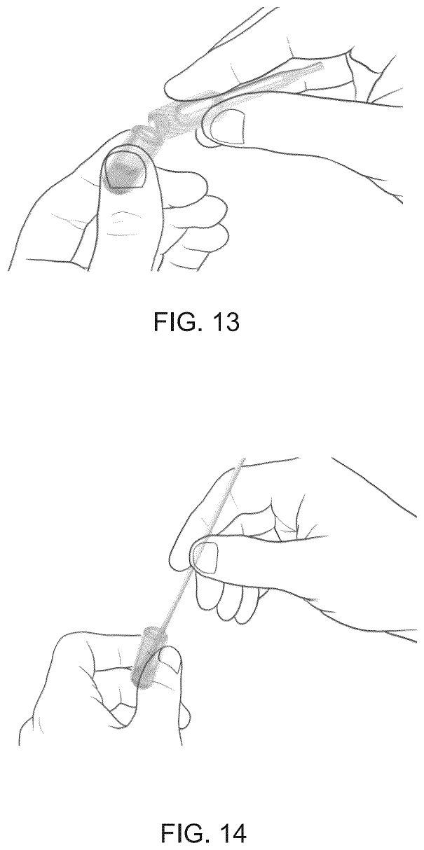

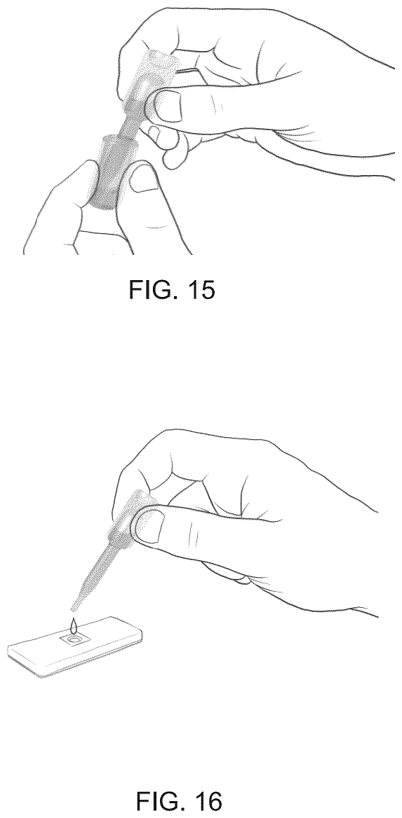

According to various embodiments of the invention, a dropper top vial 310 may be provided for collecting samples. As illustrated in FIG. 13, a blow molded dropper 317 may be twisted off, broken off, or otherwise removed from the dropper top vial 310 to open the first end 316 of the vial body 312. A sample collected with a swab 900 may be inserted through the opening in the first end 316 and mixed with a reagent or carrier fluid contained within the vial body 312 as illustrated in FIG. 14. The blow molded dropper 317 may then be used to remove the sample-containing reagent or carrier fluid from the interior of the vial body 312 as illustrated in FIG. 15. A user may squeeze the exterior of the blow molded dropper 317 and immerse the end in the reagent or carrier fluid. Release of the force on the interior space 325 creates a vacuum, pulling sample-containing reagent or carrier fluid into the interior space 325 of the blow molded dropper 317. The exterior of the blow molded dropper 317 may then be squeezed again to dispense or drop the fluid contained in the blow molded dropper 317 onto a substrate or other target for further testing as illustrated in FIG. 16.

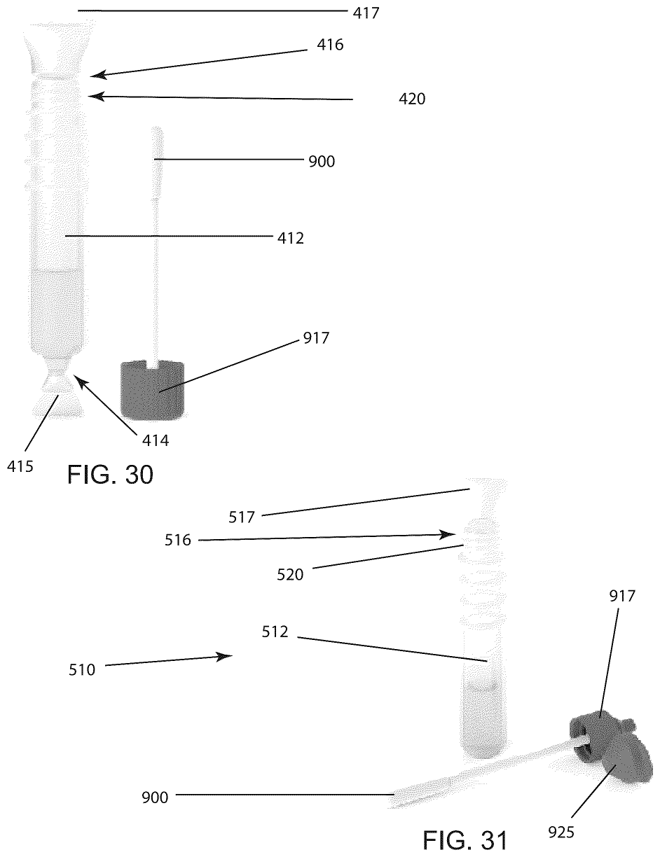

In other embodiments of the invention, a dual end vial 410 is provided for sample collection. A dual end vial 410 according to various embodiments of the invention is illustrated in FIG. 30. The dual end vial 410 includes a vial body 412 having a first end 416 and a second end 414. A first end seal 417 closes and seals the first end 416. A second end seal 215 closes and seals the second end 414. The vial body 412 includes a hollow space in which a reagent or carrier fluid may be contained. The dual end vial 410 may include one or more attachment features on an exterior surface of the vial body 412. For example, as illustrated in FIG. 30, a threads 420 may be included to allow a threaded closure to be attached to the vial body 412. In other embodiments, a snap feature, bayonet feature, or other sealing and closing feature may be used with a dual end vial 410.

In addition to the dual end vial 410, a swab 900 connected to a closure 917 may be provided for testing. The swab 900 and closure 917 combination may be used to collect a sample that is to be tested or collected with the dual end vial 410.

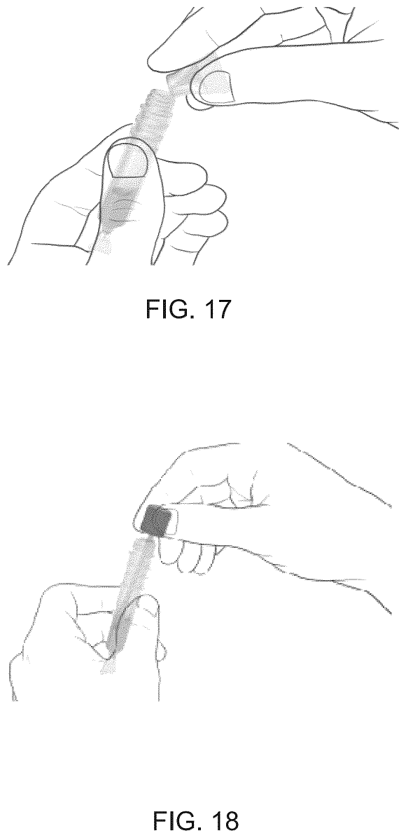

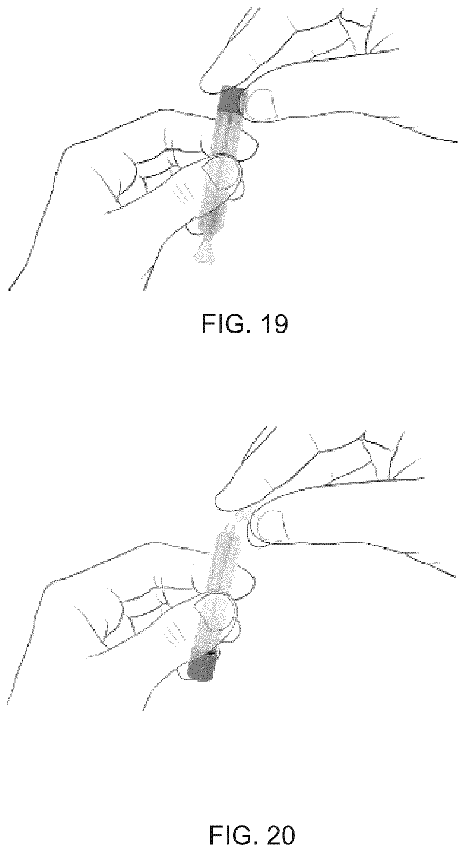

According to certain embodiments of the invention, a dual end vial 410 containing a reagent or carrier fluid therein may be used with a swab 900 and closure 917 to collect a sample. As illustrated in FIG. 17, the first end seal 417 may be removed from the dual end vial 410, opening the first end 416 and exposing an interior of the vial body 412. The swab 900 and closure 917 may be used to collect a sample and the swab 900 end inserted into the open first end 416 of the dual end vial 410 and agitated with the fluid therein as illustrated in FIG. 18. The closure 917 may be screwed on or otherwise attached to the dual end vial 410. For instance, as illustrated in FIGS. 18 and 19, the closure 917 may be screwed onto the dual end vial 410, the threads 420 of the dual end vial 410 mating with threads on an interior of the closure 917 to reseal the dual end vial 410 with the swab 900 contained in an interior space of the vial body 412.

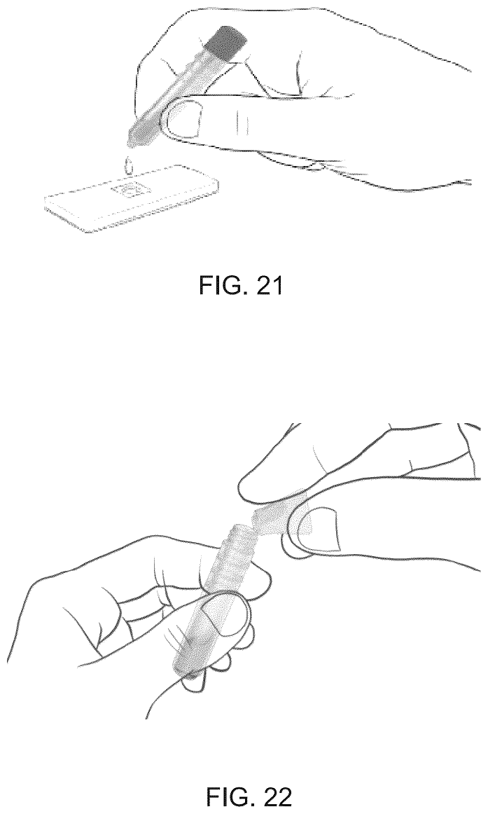

To dispense the sample mixed with the reagent or carrier fluid, the second end seal 415 may be twisted off, broken off, or otherwise removed from the second end 414 of the vial body 412 as illustrated in FIG. 20. The sample and reagent or carrier fluid may then be dispensed from the open second end 414 of the dual end vial 410 as illustrated in FIG. 21. Drops or a stream of the fluid from within the vial body 412 may be forced out of the dual end vial 410 by squeezing the vial body 412 to promote dispensing through the opening in the second end 414.

In some embodiments of the invention, a closure 917 and swab 900 combination could also be used with a threaded top vial 210 as illustrated in FIG. 28. Instead of using the top portion 217 to seal the vial body 212, the closure 917 may be connected to the threads or other connection features associated with the threaded top vial 210.

According to still other embodiments of the invention, a dropper vial 510 may be provided with a swab 900 connected to a closure 917 having a flip cap 925 and a dropper dispenser sealed by the flip cap 925 as illustrated in FIG. 31. The dropper vial 510 may include a vial body 512 having a first end 516 wherein a first end seal 517 is formed to seal the first end 516. A hollow space is defined in the vial body 512 in which a reagent or carrier fluid may be contained. One or more connection features, such as threads 520, may be present on an exterior surface of the vial body 510 adjacent to the first end 516.

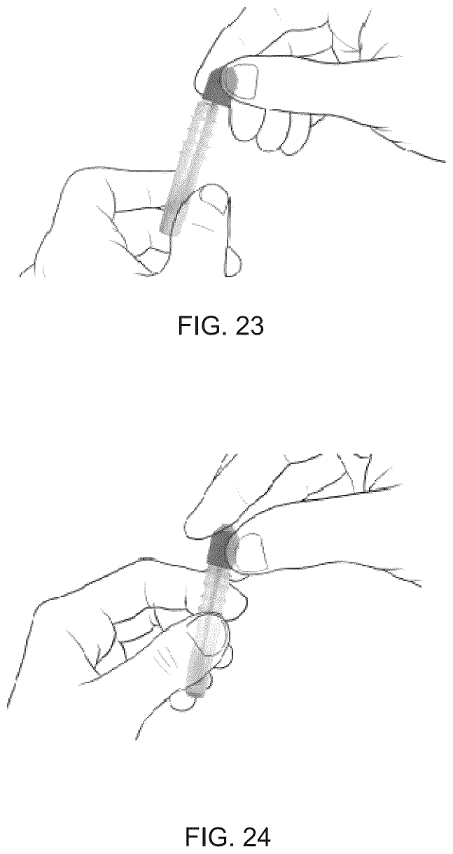

In use, the first end seal 517 of the dropper vial 510 may be removed from the dropper vial 510 as illustrated in FIG. 22. The opening formed by removal of the first end seal 517 provides sufficient space to insert a swab 900 containing a collected sample. For example, a swab 900 connected to a closure 917 having a flip cap 925 may be used to collect a sample and then the swab 900 end may be inserted into the opening of the dropper vial 510 as illustrated in FIG. 23. The swab 900 may be agitated with the reagent or carrier fluid in the vial body 512. The closure 917 attached to the swab 900 may then be connected to and sealed with the vial body 512 as illustrated in FIG. 24. For instance, the threads 520 on the exterior of the vial body 512 may engage threads on an interior of the closure 917 to secure the closure 917 to the vial body 512. Other closure features, such as snap fit closures, bayonet closures, and other closure types may also be used with embodiments of the invention.

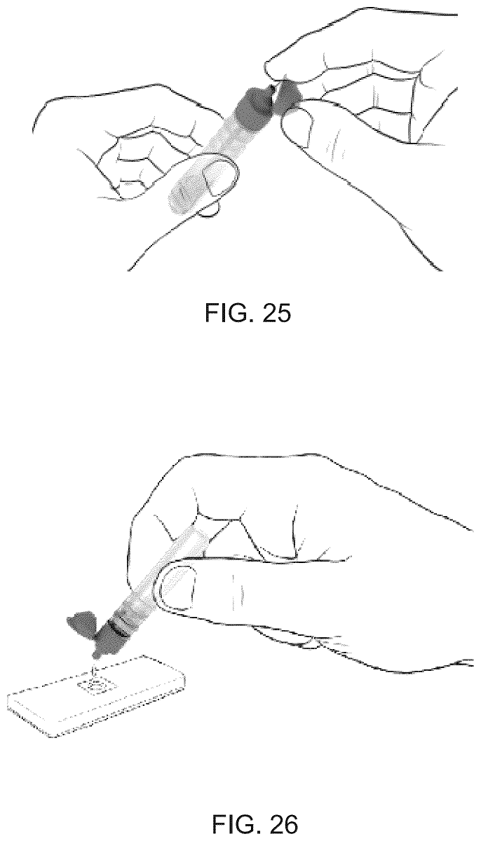

According to embodiments of the invention utilizing a swab 900, closure 917, flip cap 925 combination, the flip cap 925 of the closure 917 may be opened to expose an opening or drop dispenser opening in the closure 917 as illustrated in FIG. 25. The vial body 512 may then be squeezed to dispense sample and reagent or carrier fluid from within the vial body 512 to an exterior thereof through the closure 917. An example is illustrated in FIG. 26, wherein the flip cap 925 is opened, exposing the closure 917 opening through which sample and reagent or carrier fluid is dispensed onto a substrate for further testing.

The various vials according to embodiments of the invention may be made utilizing blow molding processes that are known in the art. In addition, blow molding and filling systems may be utilized to blow mold the vials according to embodiments of the invention and fill the vials with reagents, liquid carriers, or other products as desired.

Having thus described certain particular embodiments of the invention, it is understood that the invention defined by the appended claims is not to be limited by particular details set forth in the above description, as many apparent variations thereof are contemplated. Rather, the invention is limited only be the appended claims, which include within their scope all equivalent devices or methods which operate according to the principles of the invention as described.

What is claimed is:

1. A blow-molded, threaded top vial, comprising:
a vial body, comprising:
a first end;
a vial connection feature on an exterior surface of the vial body adjacent the first end;
a second end; and
a hollow space between the first end and the second end; and
a removable top portion attached to the first end.

2. The blow-molded, threaded top vial of claim 1, further comprising a break-away tip attached to the second end.

3. The blow-molded, threaded top vial of claim 1, wherein the second end is sealed.

4. The blow-molded, threaded top vial of claim 1, further comprising a top portion connection feature on an interior surface of the removeable top portion.

5. The blow-molded, threaded top vial of claim 4, wherein the vial connection feature comprises a vial thread on the exterior surface of the vial body;
wherein the top portion connection feature comprises a top portion thread on an interior surface of the removable top portion; and
wherein the vial thread and the top portion thread may interact to seal the top portion to the vial body.

6. The blow-molded, threaded top vial of claim 4, wherein the vial connection feature and the top portion connection feature comprise complementary connection features selected from the group consisting of thread features, bayonet features, and snap fitment features.

7. The blow-molded, threaded top vial of claim 1, further comprising a liquid in the vial body.

8. The blow-molded, threaded top vial of claim 7, wherein the liquid further comprises a reagent.

9. The blow-molded, threaded top vial of claim 7, wherein the liquid further comprises a carrier fluid.

10. The blow-molded, threaded top vial of claim 1, wherein the top portion is removed and further comprising a closure configured to attach to the vial connection feature.

11. The blow-molded, threaded top vial of claim 10, wherein the closure further comprises a closure having a connection feature selected from the group consisting of thread features, bayonet features, and snap fitment features.

12. A blow-molded, plug top vial, comprising:
a vial body, comprising:
a first end;
a second end; and
a hollow space between the first end and the second end;
a removable plug attached to the first end; and
a break-away tip attached to the second end.

13. The blow-molded, plug top vial of claim 12, further comprising a liquid in the vial body.

14. The blow-molded, plug top vial of claim 13, wherein the liquid further comprises a reagent.

15. The blow-molded, plug top vial of claim 13, wherein the liquid further comprises a carrier fluid.

16. A method of collecting and testing a biological sample, comprising:
providing a blow-molded vial, comprising:
a vial body, comprising:
a first end;
a second end; and
a hollow space between the first end and the second end;
a removable portion attached to the first end; and
a fluid in the hollow space;
removing the removable portion;
providing a swab;
collecting a sample using the swab;
inserting the swab with sample into the hollow space in the vial body through the first end so that the swab with sample contacts the fluid;
removing the swab;
sealing the first end;
providing a testing substrate; and
dispensing a portion of the sample containing fluid onto the testing substrate.

17. The method of claim 16, wherein sealing the first end further comprises sealing the first end with the removeable portion.

18. The method of claim 16, wherein providing a vial body further comprises providing a vial body comprising an attachment feature on an exterior surface of the vial body adjacent the first end.

19. The method of claim 18, wherein sealing the first end further comprises attaching the first end to the attachment feature on the exterior surface of the vial body adjacent the first end.

20. The method of claim 18, wherein the attachment feature is selected from the group consisting of a threaded feature, a bayonet feature, and a snap fitment feature.
